(19) United States
(12) Patent Application Publication
(10) Pub. No.: US 2024/0115576 A1
(43) Pub. Date: Apr. 11, 2024

(54) Caffeine Compositions

(71) Applicant: OYE Therapeutics, Inc, Crown Point, IN (US)

(72) Inventors: Brett Dines, Schererville, IN (US); Pamela A. Smith, West Lafayette, IN (US); Stephen R. Byrn, West Lafayette, IN (US); Susan Bogdanowich-Knipp, West Lafayette, IN (US)

(21) Appl. No.: 18/034,142

(22) PCT Filed: Oct. 29, 2021
(86) PCT No.: PCT/US2021/057294

§ 371 Date: Apr. 27, 2023

Related U.S. Application Data
(60) Provisional application No. 63/107,026, filed on Oct. 29, 2020.
(60) Provisional application No. 63/178,815, filed on Apr. 23, 2021.

(51) Int. Cl. A61K 31/522; A61K 9/08; A61K 31/19; A61K 31/198; A61P 39/02

(52) U.S. Cl. CPC A61K 31/522; A61K 9/08; A61K 31/19; A61K 31/198; A61P 39/02

(57) ABSTRACT

Compositions comprising, for example, caffeine, sodium benzoate and one or more of lactic acid, acetic acid, aspartic acid and glutamic acid, processes for making the compositions and methods of using the compositions. The methods include accelerating emergence from anesthesia and/or countering the effects of anesthesia, including reversing the effects of anesthesia. The methods also include treating opioid intoxication or overdose.

### CROSS-REFERENCE TO RELATED APPLICATIONS

This application claims benefit of, and priority to, U.S. Provisional Application No. 63/107,026, filed on Oct. 29, 2020, and U.S. Provisional Application No. 63/178,815, filed on Apr. 23, 2021, the entire contents of both of which are specifically incorporated by reference herein.

### FIELD OF THE DISCLOSURE

This disclosure relates to the field of caffeine compositions.

### BACKGROUND

Caffeine has potential for use in a variety of commercial applications, including pharmaceutical applications. For example, caffeine has been effective in rat studies in accelerating emergence from anesthesia when administered intravenously. The same has also been shown in humans. Anesthetic agents are known to inhibit neurotransmitter release from both neurons and secretory cells. Caffeine has been shown to elevate intracellular cAMP levels which are in turn known to facilitate neurotransmitter release. In a single-center double-blind two-way crossover trial design, participants underwent two sessions of general anesthesia administered a minimum of 2 weeks apart. A 15 mg/kg caffeine citrate infusion (corresponding to 7.5 mg/kg of caffeine base) was given and the study results showed a substantial reduction in mean time to emerge from anesthesia (16.5±3.9 (SD) min when receiving saline and 9.6±5.1 (SD) min when receiving caffeine (P=0.002)). Fong, R. et al., “Caffeine Accelerates Emergence from Isoflurane Anesthesia in Humans,” Anesthesiology, 129(5), pp. 912-920 (2018).

Caffeine and sodium benzoate have previously been prepared as a single dose vial by American Regent, Inc., for example, for use in recovery from anesthesia as reported in Warner et al., “Effects of Caffeine Administration on Sedation and Respiratory Parameters in Patients Recovering from Anesthesia,” Bosnian Journal of Basic Medical Sciences, 18(1), pp. 101-104 (2018). The standard dose has been reported as 125 mg caffeine and 125 mg sodium benzoate per milliliter. With a caffeine and sodium benzoate composition, a median dose given to patients was 150 mg of caffeine sodium benzoate with a maximum dose of 250 mg of caffeine sodium benzoate. The results of the study were inconclusive, which may be due, however, to the limited dose provided.

In accordance with the present disclosure, caffeine can also be effective in treating opioid intoxication or overdose. Worldwide, about 0.5 million deaths are attributable to drug use. More than 70% of these deaths are related to opioids, with more than 30% of those deaths caused by overdose. Naloxone, which is useful in treating overdoses, is on the World Health Organization's List of Essential Medicines. Rising rates of opioid use in the United States have contributed to an epidemic in recent decades, termed the “opioid crisis.” The United States is the highest consumer of opioids, accounting for approximately 80% of worldwide use. In 2019, an estimated 10.1 million people aged 12 or older in the USA misused opioids. Specifically, 9.7 million people misused prescription pain relievers and 745,000 people used heroin (2019 National Survey on Drug Use and Health (NSDUH)). In the midst of the opioid crisis the USA averages 128 overdose deaths per day (CDC Opioid Basics 2020). Opioid overdoses have serious adverse health consequences, even when not fatal.

In 2016, the national rate of opioid-related hospitalizations was 297 per 100,000 population, and in 2017, a total of 967,615 nonfatal drug overdoses were treated in U.S. emergency departments. Between 2012 and 2017, the rate of naloxone administration events overall (multiple administrations counted as a single event) increased 75.1%, from 573.6 to 1004.4 administrations per 100,000 emergency medical service events.

The duration of action of naloxone is 30-60 minutes. Naloxone is a non-selective and competitive opioid receptor antagonist. In treating opioid overdose, multiple doses of naloxone may be required, as the duration of action of most opioids is greater than that of naloxone and residual opioid may remain in the body should the patient's physiological ability to metabolize and or eliminate the opioid be compromised before, during, and or following overdose.

Due to the limited duration of action of naloxone for use in opioid overdose, several challenges present themselves with naloxone treatment. For example, multiple doses are often needed, which can be inopportunistic especially in a mobile transport or emergency department environment. In addition, naloxone tends to create respiratory challenges that must be monitored and/or overcome.

Caffeine's poor solubility, however, is well known. In order to be effective to act as an accelerant to emerge from anesthesia, for example, sufficient caffeine should be delivered via a suitable drug delivery route. Caffeine's solubility of 16 mg/mL at room temperature coupled with a dose of 754 mg of caffeine for a 100 kg person to emerge, for example in an indication related to anesthesia emergence (as used in clinical trials), translates into a dose of over 47 mL of volume. This volume is too large and inconvenient for bolus intravenous administration pre-surgery, during surgery, and/or for post-surgery administration by clinicians. In typical surgical suites, many medications are provided in 6 mL vials containing up to 5.25 mL of fluid for intravenous delivery. A 47 mL dose would thus require at least 9 vials, which is impractical for hospital use.

With caffeine citrate, the solubility of caffeine is about 56 mg/mL. At a dose of 15 mg caffeine citrate per kilogram of body weight, one would deliver 1500 mg caffeine citrate to 100-kilogram person or about 754 mg of caffeine. Thus, about 27 mL of solution (1500 mg/56 mg/mL) would be required to deliver such a dose, which is still an impractically large dose.

A need therefore exists for compositions with improved caffeine solubility that will allow for more practical use of caffeine in commercial applications.

### SUMMARY

In the present disclosure, compositions comprising caffeine and sodium benzoate are provided with increased solubility of caffeine so as to make feasible the use of caffeine to accelerate emergence from anesthesia or otherwise reverse the effects thereof. The compositions also make feasible the use of caffeine to treat opioid intoxication or overdose, either when administered alone or with naloxone.

In several aspects of the disclosure, compositions comprising caffeine, sodium benzoate and one or more of lactic acid, acetic acid, aspartic acid and glutamic acid are provided. In other aspects, compositions comprising an aqueous solution of caffeine and sodium benzoate, and means for increasing the solubility of caffeine in the aqueous solution are provided. Further compositions in the form of an aqueous solution and comprising caffeine at a solubility of greater than about 235 mg/mL in the solution are provided.

In further aspects of the disclosure, compositions comprising caffeine, sodium benzoate and one or more of lactic acid, acetic acid, aspartic acid and glutamic acid prepared by the process of combining caffeine, sodium benzoate and one or more of lactic acid, acetic acid, aspartic acid and glutamic acid in an aqueous solution, are provided.

In additional aspects of the disclosure, methods of accelerating emergence from anesthesia, or countering the effects of anesthesia, including reversing the effects of anesthesia, are provided comprising administering to a patient undergoing a medical procedure with an anesthesia agent a pharmaceutically effective amount of a composition of the disclosure.

In still further aspects of the disclosure, use of a pharmaceutically effective amount of a composition comprising caffeine, sodium benzoate, and one or more of lactic acid, acetic acid, aspartic acid and glutamic acid for treating a host mammal undergoing a medical procedure with an anesthesia agent is provided.

In yet additional aspects of the disclosure, use of a pharmaceutically effective amount of a composition comprising caffeine, sodium benzoate, and one or more of lactic acid, acetic acid, aspartic acid and glutamic acid for accelerating emergence from anesthesia or countering the effects of anesthesia, including reversing the effects of anesthesia, in a host mammal is provided.

In still further aspects of the disclosure, kits comprising caffeine, sodium benzoate, one or more of lactic acid, acetic acid, aspartic acid and glutamic acid, and optionally water are provided.

In additional aspects of the disclosure, stable compositions are provided.

In further aspects of the disclosure, methods of treating patients who suffer from opioid toxicity or overdose comprising treating a patient in need thereof with a pharmaceutically effective amount of a composition of the disclosure are provided.

In still further aspects of the disclosure, use of a pharmaceutical composition comprising caffeine, sodium benzoate, and one or more of lactic acid, acetic acid, aspartic acid and glutamic acid for treating a host mammal undergoing treatment for opioid toxicity or overdose is provided.

More embodiments are included in the detailed description that follows.

### DETAILED DESCRIPTION

Various additional embodiments of the disclosure will now be explained in further detail. Both the foregoing general description and the following detailed description are exemplary and explanatory only, and are not restrictive of this disclosure or of the claims. Any discussion of certain embodiments or features, including those depicted in the Examples, serve to illustrate certain exemplary aspects of the disclosure. The disclosure and claims are not limited to the embodiments specifically discussed herein.

The disclosure includes compositions comprising caffeine, sodium benzoate, and one or more of lactic acid, acetic acid, aspartic acid and glutamic acid. The compositions may also comprise water and may be in the form of a solution, such as an aqueous solution.

Also included are compositions comprising: an aqueous solution of caffeine and sodium benzoate, and means for increasing the solubility of caffeine in the aqueous solution. The composition itself may also be an aqueous composition. An exemplary means for increasing the solubility of caffeine in the aqueous solution of caffeine and sodium benzoate is lactic acid. Additional exemplary means for increasing the solubility of caffeine in the aqueous solution of caffeine and sodium benzoate are acetic acid, aspartic acid and glutamic acid.

Also included is a composition in the form of an aqueous solution and comprising caffeine at a solubility of greater than about 235 mg/mL in the solution. Such a composition may further comprise sodium benzoate. The composition may also comprise, for example, one or more of lactic acid, acetic acid, aspartic acid and glutamic acid.

Compositions of the disclosure also include those comprising caffeine, sodium benzoate, and one or more C₂ to C₅ carboxylic acids, such as lactic acid, aspartic acid, acetic acid or glutamic acid. Further compositions comprise caffeine, sodium benzoate, and one or more C₄ to C₅ amino acids, such as aspartic acid and glutamic acid. Yet further compositions comprise caffeine, sodium benzoate, and one or more C₂ to C₅ carboxylic acid alcohols, such as lactic acid. Still further compositions comprise one or more C₂ to C₅ dicarboxylic acids, such as aspartic acid and glutamic acid. Any such compositions may further comprise water and may be in the form of a solution.

Compositions of the disclosure have improved caffeine solubility and can be stable compositions. In many embodiments, caffeine is added to a solution of sodium benzoate and upon dissolution, additional water is added followed by adding, for example, one or more of lactic acid, acetic acid, aspartic acid and glutamic acid last. Due to improved solubility, compositions of the disclosure are capable of commercial applications such as to deliver caffeine for accelerating emergence from anesthesia, or otherwise overcoming the effects of anesthesia, including reversing the effects of anesthesia. The compositions are also capable of treating opioid intoxication or overdose, either alone or together with administration of naloxone.

When measuring solubility of caffeine in the compositions of the disclosure, solids were generally added to dissolution and increasing amounts of water were added until all solids went into solution. In several examples (e.g., RP1-49-2; RP1-49-3; RP1-49-4; RP1-49-5; and RP1-57-5) the concentration of the caffeine in solution was not a solubility measurement, but simply represented the amount of caffeine in the solution with the specified amounts of input materials. Such solutions were undersaturated with respect to caffeine. In other examples, (e.g., RP1-60-1; RP1-60-2; and RP1-62-4), slurries were made, and water was added until the solids dissolved. Thus, these concentrations more closely reflect the solubility of caffeine.

By combining lactic acid, for example, with caffeine and sodium benzoate in various combinations, higher caffeine solubilities were achieved, such as, in RP1-62-4, where a solubility of 396 mg/mL was measured, than caffeine solubilities previously reported. Thus, in these and other embodiments, the solubility of caffeine in compositions of the disclosure, including those comprising caffeine, sodium benzoate, one or more of lactic acid, acetic acid, aspartic acid and glutamic acid and water, may be greater than about 235 mg/mL, or 236 mg/ml or greater, or 237 mg/ml or greater, or 238 mg/ml or greater, or 240 mg/ml or greater, or 250 mg/ml or greater, or greater than 250 mg/ml. Examples include from about 240 mg/mL to about 500 mg/mL, from about 300 mg/mL to about 450 mg/mL, from about 300 gm/mL to about 400 mg/mL, from about 340 mg/mL to about 420 mg/mL, from about 360 mg/mL to about 400 mg/mL, from about 250 mg/ml to about 400 mg/ml, from about 350 mg/ml to about 400 mg/ml, from about 260 mg/ml to about 500 mg/ml and from about 260 mg/ml to about 400 mg/ml.

Stability of caffeine compositions is also described herein. At least some compositions of the disclosure are stable against formation of precipitates. For example, at least some compositions exhibit no precipitation after a duration of at least one week, such as under ambient temperature (such as from 20° C. to 22° C.) or at the temperature of an operating room (such as from 12° C. to 18° C.). In some embodiments, there has been no precipitation under these temperatures or even under refrigerated conditions (such as, for example, from 3° C. to 5° C. or at −2° C.) for more than eight weeks. In some embodiments, there is no precipitation for more than three months, whether or not the composition has been under refrigerated conditions.

In additional embodiments, at least some compositions of the disclosure are heat stable when subjected to a temperature of 135° C. for 45 minutes. Heat stability can be assessed, for example, by an HPLC chemical stability evaluation. The absence of any additional extraneous peaks in the composition after exposure to these conditions reveals that the components in the composition have not broken down into other chemical species. Heat stability under these conditions indicates that the compositions should be capable of being terminally sterilized.

In the disclosure, solubility and concentration measurements are used with the word “about” to describe the measurements. For example, with respect to solubilities, the term represents variability introduced by the measurement technique which in this case is on the order of 0 to 2%. When values or ranges mentioned for solubility, concentration or other indicated parameters are modified by the term “about,” the exact values or ranges also form embodiments of the disclosure.

Tables 1A, 1B, and 1C provide certain data parameters for 8 examples of compositions of caffeine, sodium benzoate and water. For 7 materials, lactic acid was also included. The methods by which the compositions were made are set forth in Example 2 and Example 3. In each composition, the mass or volume of materials used, the molecular weights of each component, the concentration of lactic acid used, and the lactic acid density are provided. The relative compositional makeup by mass and equivalents is also calculated therein and the pH reported.

Formula RPI-57-5 is a composition comprising caffeine and sodium benzoate in equivalent mass amounts and no lactic acid is added. As indicated, the solubility found was 235 mg of caffeine per mL. For RP1-49-2; -3; -4; and -5; the concentration of caffeine was found to be 250 mg/mL, but there was no attempt in these compositions to identify the limiting amount of caffeine. Thus, these compositions are undersaturated. By comparison, saturation conditions were attempted and approached in RP1-60-1; -2; and RP1-62-4 and the maximum concentration achieved was about 396 mg/mL of caffeine. At such a concentration, only 1.9 mL of solution are needed to dose a 100 kg human at 7.5 mg/kg (754 mg caffeine/396 mg/mL). This not only minimizes the overall volume needed, but also minimizes the mass of inactive ingredients delivered, making such a concentration feasible for commercial use medically and in foodstuffs.

TABLE 1A

| | | | | | Lactic | | | |
|---|---|---|---|---|---|---|---|---|
| | Sodium | Sodium | | | acid | Lactic | | Total |
| | Benzoate | Benzoate | Caffeine | Caffeine | solution | acid | H₂O | Solution |
| Composition | (mg) | (eq) | (mg) | (eq) | (mL) | (eq) | (mL) | (mL) |
| RP1-49-2 | 2411.8 | 1.300 | 2500.1 | 1.000 | 0.232 | 0.195 | 9.768 | 10.00 |
| RP1-49-3 | 2504.8 | 1.350 | 2500.4 | 1.000 | 0.240 | 0.202 | 9.760 | 10.00 |
| RP1-49-4 | 2597.4 | 1.400 | 2500.1 | 1.000 | 0.249 | 0.209 | 9.751 | 10.00 |
| RP1-49-5 | 2690.3 | 1.450 | 2500.1 | 1.000 | 0.258 | 0.217 | 9.742 | 10.00 |
| RP1-60-1 | 2597.5 | 1.400 | 2500.3 | 1.000 | 0.249 | 0.209 | 7.75 | 7.999 |
| RP1-60-2 | 2690.2 | 1.450 | 2500.1 | 1.000 | 0.258 | 0.217 | 7.5 | 7.758 |
| RP1-62-4 | 2690.5 | 1.450 | 2500.2 | 1.000 | 0.310 | 0.261 | 6 | 6.310 |
| RP1-57-5 | 2350.2 | 1.348 | 2350.1 | 1.000 | 0.000 | 0.000 | 10 | 10.00 |

TABLE 1B

| | | | | | Lactic | | | |
|---|---|---|---|---|---|---|---|---|
| | | | | | acid | H₂O | Mass | Mass |
| | | | | | from | from | ratio of | ratio of |
| | | | | | Lactic | Lactic | Caffeine | Caffeine |
| | % | | % | | acid | acid | to | to |
| | Sodium | % | Lactic | % | solution | solution | Sodium | Lactic |
| Composition | Benzoate | Caffeine | acid | H₂O | (mg) | (mg) | Benzoate | Acid |
| RP1-49-2 | 16.148 | 16.739 | 1.516 | 65.597 | 226.391 | 29.505 | 1.037 | 11.043 |
| RP1-49-3 | 16.665 | 16.636 | 1.558 | 65.140 | 234.198 | 30.522 | 0.998 | 10.676 |
| RP1-49-4 | 17.175 | 16.532 | 1.607 | 64.687 | 242.980 | 31.667 | 0.963 | 10.289 |
| RP1-49-5 | 17.680 | 16.430 | 1.654 | 64.236 | 251.763 | 32.811 | 0.929 | 9.930 |
| RP1-60-1 | 19.794 | 19.054 | 1.852 | 59.300 | 242.980 | 31.667 | 0.963 | 10.290 |
| RP1-60-2 | 20.734 | 19.269 | 1.940 | 58.057 | 251.763 | 32.811 | 0.929 | 9.930 |
| RP1-62-4 | 23.329 | 21.679 | 2.623 | 52.368 | 302.505 | 39.425 | 0.929 | 8.265 |
| RP1-57-5 | 15.987 | 15.987 | 0.000 | 68.026 | 0.000 | 0.000 | 1.000 | 0.000 |

TABLE 1C

| | | | Molar | | | | |
|---|---|---|---|---|---|---|---|
| | | | ratio of | | Molar | | |
| | | | Sodium | | ratio of | Amount of | |
| | | Sodium | Benzoate | Lactic | Lactic | Caffeine | |
| | Caffeine | Benzoate | to | Acid | acid to | dissolved | |
| Composition | (mmol) | (mmol) | Caffeine | (mmol) | Caffeine | (mg/mL) | pH |
| RP1-49-2 | 12.875 | 16.736 | 1.300:1 | 2.513 | 0.195:1 | 250.010 | 5.860 |
| RP1-49-3 | 12.876 | 17.381 | 1.350:1 | 2.600 | 0.202:1 | 250.040 | 5.870 |
| RP1-49-4 | 12.875 | 18.024 | 1.400:1 | 2.697 | 0.209:1 | 250.010 | 5.940 |
| RP1-49-5 | 12.875 | 18.668 | 1.450:1 | 2.795 | 0.217:1 | 250.010 | 5.920 |
| RP1-60-1 | 12.876 | 18.024 | 1.400:1 | 2.697 | 0.209:1 | 312.577 | 5.87 |
| RP1-60-2 | 12.875 | 18.668 | 1.450:1 | 2.795 | 0.217:1 | 322.261 | 5.94 |
| RP1-62-4 | 12.875 | 18.670 | 1.450:1 | 3.358 | 0.261:1 | 396.228 | 5.91 |
| RP1-57-5 | 12.102 | 16.308 | 1.348:1 | 0.000 | 0.000 | 235.010 | 8.01 |

Caffeine molar mass: 194.19 g/mol
Sodium Benzoate molar mass: 144.11 g/mol
Lactic Acid molar mass: 90.08 g/mol
The density of the Lactic Acid Solution Used was measured to be 1.103 g/mL
The concentration of the Lactic Acid Solution was 88.47% in Water

Compositions of the disclosure may include, for example, molar ratios of caffeine to sodium benzoate from about 1:1.0 to about 1:1.5 caffeine to sodium benzoate, such as from about 1:1.0 to about 1:1.45, or from about 1:1.3 to about 1:1.5, or from about 1:1.3 to about 1:1.45, including 1:1.1, 1:1.15, 1:1.2, 1:1.25 1:1.3, 1:1.35, 1:1.4, 1:1.45, and 1:1.5. In these and other embodiments, the molar ratio of caffeine to the total moles of lactic acid, acetic acid, aspartic acid and glutamic acid may be from about 1:0.1 to about 1:0.3, including 1:0.11, 1:0.12, 1:0.13, 1:0.14, 1:0.15, 1:0.16, 1:0.17, 1:0.18. 1:0.19, 1:0.2, 1:0.21, 1:0.22, 1:0.23, 1:0.24, 1:0.25, 1:0.26, 1:0.27, 1:0.28, 1:0.29 and 1:0.3. The compositions may also comprise a molar ratio of sodium benzoate to the total moles of lactic acid, acetic acid, aspartic acid and glutamic acid of from about 1:0.15 to about 1:0.18.

The pH of the compositions of the disclosure are often from about 5 to about 6, such as from about 5.80 to about 5.92, or from about 5.70 to about 5.90, including of about 5.1, 5.2, 5.3, 5.4, 5.5, 5.6, 5.7, 5.8, and about 5.9. Further examples of pH include about 5.80, 5.81, 5.82, 5.83, 5.84, 5.85, 5.86, 5.87, 5.88, 5.89, 5.90, 5.91, and about 5.92.

At least some compositions of the disclosure may contain from about 15% to about 25% caffeine by weight, from about 15% to about 25% by weight sodium benzoate, from about 1% to about 3% by weight lactic acid, and from about 50% to about 70% by weight water. Such ranges may further include from about 15% to about 22% caffeine by weight, from about 16% to about 24% sodium benzoate by weight, from about 1.4% to about 1.8% lactic acid by weight, and from about 50% to about 66% water by weight.

In many embodiments, the weight percent of sodium benzoate is greater than the weight percent of caffeine. In many embodiments the difference between the weight percent of sodium benzoate and the weight percent of caffeine is from about 1% to about 2%. By way of example, in a composition containing 2690.5 mg of sodium benzoate, 2500.2 mg of caffeine, 302.505 mg of lactic acid, and water to make a total volume of 6.3 mL, the weight percent of sodium benzoate is 23.3% and that of caffeine 21.7%, the difference in their weight percents being about 1.6%.

In some embodiments, compositions of the disclosure comprise less than 14 wt % lactic acid (or alternatively the combined wt % of lactic acid, acetic acid, aspartic acid and glutamic acid), such as from about 1% to about 3% lactic acid by weight. These and other compositions can comprise more than 30 wt % water, such as more than 50 wt % water. The compositions may also comprises a higher wt % of caffeine than lactic acid (or alternatively the combined wt % of lactic acid, acetic acid, aspartic acid and glutamic acid).

A further embodiment of a composition of the disclosure comprises caffeine, sodium benzoate, lactic acid and water;
wherein the composition is in the form of an aqueous solution;
wherein the molar ratio of caffeine to sodium benzoate is from about 1:1.3 to about 1:1.45;
wherein the molar ratio of sodium benzoate to lactic acid is from about 1:0.15 to about 1:0.18; and
wherein the solubility of the caffeine in the aqueous solution is 250 mg/mL or greater.

When formulated, the compositions of the disclosure may be in a solid dry state, and then optionally reconstituted with water. Compositions in a solid dry state could themselves be administered to a patient in accordance with the methods of the disclosure. For example, they could be made into a thin film and administered to a patient in that form.

In some embodiments, the compositions of the disclosure are pharmaceutical compositions, which can optionally comprise one or more pharmaceutically acceptable excipients. Exemplary excipients include preservatives, antioxidants and buffers, including any combinations of two or more of these. Example preservatives include paraben and parabens. Example antioxidants include ascorbate, bisulfite and thioglycerol. Example buffers include bicarbonate, phosphate and tromethamine.

The amount of caffeine dosed may be related to the weight of the individual being treated. This could include doses from about 2.5 mg caffeine/kg of body weight to about 15 mg caffeine/kg of body weight, such as 7.5 mg/kg, 10 mg/kg or 12.5 mg/kg. Thus, the dose may range, for example from about 250 mg caffeine to about 1200 mg of caffeine. One such dose is about 750 mg of caffeine.

The disclosure includes methods of delivering a composition of the disclosure (typically aqueous and in the form of a solution) to a patient for the purposes of accelerating emergence from anesthesia or otherwise countering the effects of anesthesia such as by reversing the effects of anesthesia. Earlier developments of caffeine for such effects have been hampered by poor solubility. Compositions of the disclosure include those with improved caffeine solubility. The caffeine compositions (such as pharmaceutical compositions) may be given before, concurrent, or after the delivery of anesthesia.

Typical anesthesia agents are those that may be given for surgery or other medical procedures. Anesthetics may be inhaled or delivered intravenously. Inhaled anesthetic gases include halothane, isoflurane, enflurane, desflurane, and sevoflurane. Other anesthetics may also be used other than inhalants. NMDA receptor agonists such as ketamine or alpha 2 agonists such as dexmedetomidine may be used. Other examples include etomidate, lidocaine, magnesium, forskolin, theophylline, methylphenidate, preladenant or various combinations thereof such as preladenant and forskolin. Another example is propofol. Opioids such as fentanyl, remifentanil, morphine, and hydromorphone may also be used.

When delivered intravenously, for example, the dosage may be given as a bolus dose, as an infusion, or via a drip line or via smaller intravenous bags such as 50 mL bags. When the dosage is provided as an IV infusion, for example, it may be administered over any appropriate period of time, such as over a period of 10 minutes. The dosing may occur concurrent with, before or after administration of an anesthesia agent, such as 10 minutes before the end of surgery. For example, the composition could be administered at any appropriate point during the surgery, or post-surgery in a recovery room, after induction or intubation of a patient at the beginning of surgery, or at any time throughout a surgery procedure to mitigate common anesthesia adverse events such as cardiac depression and hemodynamic instability.

The emergence from anesthesia typically then occurs faster than without the caffeine compositions of the disclosure. In many embodiments the administration of the composition of the disclosure does not wake the patient any faster from anesthesia unless the patient has stopped receiving the anesthesia agent. The emergence may, for example, occur up to and including 30 minutes faster, 15 minutes faster, up to and including 15 minutes faster, from 1 minute to 30 minutes faster, or from 15 minutes to 30 minutes faster. In these and other embodiments, one or more pharmaceutically acceptable excipients may be added to the compositions. For example, excipients that may be used in parenteral compositions, such as intravenous compositions, may be added.

In many embodiments, there is no alcohol in the compositions of the disclosure. Such alcohols include polyols such as glycols. Polyethylene glycol is an example of such a glycol.

The methods of the disclosure may be applied to accelerate emergence from anesthesia or otherwise counter the effects of anesthesia, such as reverse the effects of anesthesia to a human or non-human vertebrate mammal. Non-human examples include companion animals such as dogs and cats as well as agricultural animals such as horses, cows, and pigs.

Given the increased solubility of caffeine in the compositions of the disclosure, various routes of caffeine delivery in the methods of the disclosure may be used to deliver the compositions. These include by parenteral, intravenous, intramuscular, subcutaneous, and oral routes such as buccal or oromucosal routes. In some embodiments, the compositions of the disclosure, including pharmaceutical compositions of the disclosure, are not in an oral dosage form. In some embodiments, the composition of the disclosure is not included in a capsule. For example, in some embodiments the composition of the disclosure is not in the form of an aqueous solution inside of a capsule.

Further embodiments therefore include a parenteral pharmaceutical composition comprising a composition of the disclosure. The parenteral composition may have any appropriate dose volume, such as from about 2 to about 8 mL. The composition could be configured for intravenous administration, or for intramuscular or subcutaneous routes.

Embodiments also include use of a pharmaceutical composition of the disclosure for treating a host mammal undergoing a medical procedure with an anesthesia agent. The pharmaceutical composition could comprise one or more pharmaceutically acceptable excipients. Such a composition could optionally not be an oral dosage form. The pharmaceutical composition could be, for example, an intravenous dosage form and the host mammal could be human. Alternatively, the host mammal could be a non-human mammal.

The disclosure also includes use of a pharmaceutical composition of the disclosure for accelerating emergence from anesthesia or countering the effects of anesthesia in a host mammal, such as in a human. The method of countering the effects of anesthesia could be, for example, reversing the effects of anesthesia. The pharmaceutical composition could comprise one or more pharmaceutically acceptable excipients. The composition could optionally not be an oral dosage form. The pharmaceutical composition could be, for example, an intravenous dosage form and the host mammal could be human. Alternatively, the host mammal could be a non-human mammal.

Compositions of the disclosure may also be provided to patients who have been dosed with naloxone to help overcome the limitations of naloxone due to caffeine's ability to stimulate respiratory response, and the duration of action may be extended by caffeine administration for up to 4-6 hours following administration. Without being bound by theory, it is believed that caffeine, as a bronchial smooth muscle relaxant, a CNS stimulant, a cardiac muscle stimulant, and a diuretic, is able to counter the deleterious effects of opioid overdose by itself or in combination with naloxone.

Opioid overdose often results in the presence of acute and or recurrent respiratory depression requiring repeat administration of naloxone. Respiratory depression (i.e., respiratory rate <12 breaths/minute or apnea in the absence of physiological sleep) and oxygen saturation <90% on room air are suggestive of opioid toxicity, particularly when they occur in conjunction with stupor and/or miosis. Naloxone administered under the current standard of care results in the competitive antagonism of opioid receptors that lead to respiratory depression, diminished respiratory rate, and or apnea. The care of patients who are opioid intoxicated or overdose with long-acting or extended-release opioids frequently have recurrent respiratory depression. Initial management of opioid toxicity involves maintaining adequate oxygenation through ventilation (respiratory rate >12 breaths per minute and oxygen saturation >90%). The inability to support a patient's airway, breathing, and circulation can lead to negative outcomes, morbidity and mortality.

The administration of caffeine, including caffeine in a composition of the disclosure, may be used to manage opioid intoxication or overdose and mitigate respiratory depression, diminished respiratory rate, and or apnea. Another embodiment of the disclosure therefore includes a method of treating a patient suffering from opioid intoxication or overdose comprising administering to the patient a pharmaceutically effective amount of a composition of the disclosure (such as a pharmaceutical composition), including parenteral administration. Such administration can include dosages from about 2.5 mg caffeine/kg of body weight to about 15 mg caffeine/kg of body weight, including dosages previously mentioned for administering compositions of the disclosure. Such administration may be done in lieu of or in addition to dosing with naloxone and, in some embodiments, this reduces the amount of naloxone needed for clinical benefit.

Opioid intoxication is often associated with or directly due to dosing errors for approved drugs. If a patient increases the appropriate dose or duration for opioid administration beyond what is acceptable, the resulting opioid toxicity can lead to complications and adverse events. Overdose is often associated with illegal drug use and consumption. Overdose can compromise life-sustaining systems, such as a properly-maintained heartbeat and respiration, and can have potential life-threatening consequences such as respiratory arrest. Example opioids include oxycodone, hydrocodone, morphine, methadone, fentanyl and heroin.

The compositions of the disclosure may be dosed prior to, after, or concurrently with naloxone. In some embodiments, the dosing of caffeine compositions of the disclosure to patients suffering from opioid to intoxication or overdose mitigates pulmonary edema, including, for example, by increasing diuresis. In many embodiments, the caffeine compositions of the disclosure stimulate the myocardium and increase heart rate, cardiac output, and stroke volume, and or mean arterial blood pressure, when dosed to a patient suffering from opioid intoxication or overdose. The route of administration may be, for example, intravenous, intramuscular, subcutaneous. Administration of the composition may be through a bolus dose or via infusion or a drip line.

Opioid withdrawal may occur, due in part, to naloxone administration. In many embodiments of the disclosure, administration of caffeine compositions of the disclosure to patients suffering from opioid intoxication or overdose and who have received naloxone limits the precipitation of physiological symptoms of opioid withdrawal. The management of such symptoms may be achieved because caffeine targets the opposite physiological effects of the opioid such as reducing noradrenergic stimulation of the brain via central nervous system stimulation and reduction of the effects of hypotension.

Opioid intoxication or overdose may also be due to polysubstance intoxications such as those including benzodiazepines. Caffeine compositions of the disclosure may also be deployed with compounds other than naloxone to treat opioid intoxication or overdose. Such compounds include opioid-agonist tapering or alpha2-adrenergic agonists.

The disclosure also includes use of a pharmaceutical composition of the disclosure for treating a host mammal suffering from opioid intoxication or overdose. The pharmaceutical composition may comprise water and/or one or more pharmaceutically acceptable excipients. Such a composition could optionally not be an oral dosage form. The pharmaceutical composition could be, for example, an intravenous dosage form and the host mammal could be human. Alternatively, the host mammal could be a non-human mammal.

The disclosure also includes processes for preparing caffeine pharmaceutical compositions. Such processes could comprise combining water with sodium benzoate to form a solution; treating the sodium benzoate solution with caffeine, and then adding one or more of lactic acid, acetic acid, aspartic acid and glutamic acid to form a caffeine pharmaceutical composition. The added one or more of lactic acid, acetic acid, aspartic acid and glutamic acid could optionally be in an aqueous solution. In some embodiments, the pharmaceutical composition is an intravenous dosage form.

The caffeine pharmaceutical compositions prepared by the processes form additional embodiments of the disclosure. The compositions may comprise one or more pharmaceutically acceptable excipients. The compositions could also be configured for any appropriate route of administration, such as intravenous, intramuscular, subcutaneous, buccal, or oromucosal delivery.

Other applications for compositions of the disclosure include caffeine for beverages, food and candy, including confectionaries. Because caffeine is bitter, reducing the volume of caffeine in a product allows for increased volume of sweeteners or other non-bitter components so as to improve overall flavor of the product.

A further embodiment of the disclosure therefore includes a beverage, food or candy, such as for oral consumption by a mammal, which comprises a composition of the disclosure. The foods include baked foods made from flour, such as a cake, pastry, doughnut or cookie. This includes such foods having one or more toppings or an icing that comprises compositions of the disclosure. The foods also include chocolates, and the candies include chewing gum and breath fresheners. Exemplary beverages include carbonated beverages, tea, coffee and energy drinks.

Compositions of the disclosure could be included in an intermediate product during preparation of the beverage, food or candy, as a flavor enhancement additive for bitterness or odor suppression, or for otherwise making the intermediate or final product more palatable. The compositions could also be added after the beverage, food or candy has been prepared.

### Exemplary Embodiments

Further exemplary embodiments of the disclosure include those provided in the following non-limiting clauses.

Clause 1. A composition comprising caffeine, sodium benzoate, and one or more of lactic acid, acetic acid, aspartic acid and glutamic acid.

Clause 2. The composition of clause 1, further comprising water.

Clause 3. The composition of clause 2, wherein the composition is in the form of a solution.

Clause 4. A composition comprising:
an aqueous solution of caffeine and sodium benzoate, and
means for increasing the solubility of caffeine in the aqueous solution.

Clause 5. A composition in the form of an aqueous solution and comprising caffeine at a solubility of greater than about 235 mg/mL in the solution.

Clause 6. The composition of clause 5, which comprises sodium benzoate.

Clause 7. The composition of any one of clauses 1-6, which comprises lactic acid.

Clause 8. The composition of any one of clauses 1-7, which comprises acetic acid.

Clause 9. The composition of any one of clauses 1-8, which comprises glutamic acid.

Clause 10. The composition of any one of clauses 1-9, which comprises aspartic acid.

Clause 11. The composition of any one of clauses 1-6, which comprises two or more of lactic acid, acetic acid, aspartic acid and glutamic acid.

Clause 12. The composition of any one of clauses 1-11, which comprises sodium benzoate, and wherein the molar ratio of caffeine to sodium benzoate is from about 1:1.0 to about 1:1.5.

Clause 13. The composition of clause 12, wherein the molar ratio of caffeine to sodium benzoate is from about 1:1.0 to about 1:1.45.

Clause 14. The composition of clause 12, wherein the molar ratio of caffeine to sodium benzoate is from about 1:1.3 to about 1:1.50.

Clause 15. The composition of clause 12, wherein the molar ratio of caffeine to sodium benzoate is from about 1:1.3 to about 1:1.45.

Clause 16. The composition of clause 12, wherein the molar ratio of caffeine to sodium benzoate is about 1:1.4.

Clause 17. The composition of clause 12, wherein the molar ratio of caffeine to sodium benzoate is about 1:1.45.

Clause 18. The composition of any one of clauses 1-17, which comprises one or more of lactic acid, acetic acid, aspartic acid and glutamic acid, and wherein the molar ratio of caffeine to the total moles of lactic acid, acetic acid, aspartic acid and glutamic acid is from about 1:0.1 to about 1:0.3.

Clause 19. The composition of clause 18, wherein the molar ratio of caffeine to the total moles of lactic acid, acetic acid, aspartic acid and glutamic acid is about 1:0.2.

Clause 20. The composition of clause 18, wherein the molar ratio of caffeine to the total moles of lactic acid, acetic acid, aspartic acid and glutamic acid is about 1:0.21.

Clause 21. The composition of clause 18, wherein the molar ratio of caffeine to the total moles of lactic acid, acetic acid, aspartic acid and glutamic acid is about 1:0.22.

Clause 22. The composition of clause 18, wherein the molar ratio of caffeine to the total moles of lactic acid, acetic acid, aspartic acid and glutamic acid is about 1:0.26.

Clause 23. The composition of any one of clauses 1-22, which comprises sodium benzoate and one or more of lactic acid, acetic acid, aspartic acid and glutamic acid, and wherein the molar ratio of sodium benzoate to the total moles of lactic acid, acetic acid, aspartic acid and glutamic acid is from about 1:0.15 to about 1:0.18.

Clause 24. The composition of any one of clauses 3-23, wherein the pH of the composition is from about 5 to about 6.

Clause 25. The composition of clause 24, wherein the pH is from about 5.80 to about 5.92.

Clause 26. The composition of clause 24, wherein the pH is from about 5.70 to about 5.90.

Clause 27. The composition of any one of clauses 2-4 and 6-26, wherein the solubility of the caffeine is greater than about 235 mg/mL.

Clause 28. The composition of any one of clauses 2-27, wherein the solubility of the caffeine is 236 mg/ml or greater.

Clause 29. The composition of any one of clauses 2-28, wherein the solubility of the caffeine is 238 mg/ml or greater.

Clause 30. The composition of clause 29, wherein the solubility of the caffeine is 240 mg/ml or greater.

Clause 31. The composition of clause 29, wherein the solubility of the caffeine is 250 mg/mL or greater.

Clause 32. The composition of clause 29, wherein the solubility of the caffeine is from about 240 mg/mL to about 500 mg/mL.

Clause 33. The composition of clause 29, wherein the solubility of the caffeine is from about 300 mg/mL to about 400 mg/mL.

Clause 34. The composition of clause 29, wherein the solubility of the caffeine is from about 300 mg/mL to about 450 mg/mL.

Clause 35. The composition of clause 29, wherein the solubility of the caffeine is from about 340 mg/mL to about 420 mg/mL.

Clause 36. The composition of clause 29, wherein the solubility of the caffeine is from about 360 mg/mL to about 400 mg/mL.

Clause 37. The composition of any one of clauses 2-3, wherein the weight percent of caffeine is from about 15% to about 25%, the weight percent of sodium benzoate is from about 15% to about 25%, the weight percent of lactic acid is from about 1% to about 3%, and the weight percent of water is from about 50% to about 70%.

Clause 38. The composition of clause 37, wherein the weight percent of caffeine is from about 15% to about 22%, the weight percent of sodium benzoate is from about 16% to about 24%, the weight percent of lactic acid is from about 1.4% to about 1.8%, and the weight percent of water is from about 50% to about 66%.

Clause 39. The composition of any one of clauses 1-36, which comprises less than 14 wt % lactic acid.

Clause 40. The composition of clause 39, which comprises from about 1% to about 3% lactic acid by weight.

Clause 41. The composition of any one of clauses 1-36 and 39-40, which comprises more than 30 wt % water.

Clause 42. The composition of clause 41, which comprises 50 wt % or more water.

Clause 43. The composition of any one of clauses 1-36 and 39-42, which comprises a higher wt % of caffeine than lactic acid.

Clause 44. The composition of any one of clauses 1-43, which comprises sodium benzoate, and wherein the difference between the weight percent of sodium benzoate and caffeine is from 1% to 2% by weight.

Clause 45. A composition comprising caffeine, sodium benzoate, lactic acid and water;
wherein the composition is in the form of an aqueous solution;
wherein the molar ratio of caffeine to sodium benzoate is from about 1:1.3 to about 1:1.45;
wherein the molar ratio of sodium benzoate to lactic acid is from about 1:0.15 to about 1:0.18; and
wherein the solubility of the caffeine in the aqueous solution is 250 mg/mL or greater.

Clause 46. The composition of any one of clauses 2-45, wherein the composition is stable against formation of precipitates.

Clause 47. The composition of clause 46, which is in the form an of an aqueous solution and exhibits no precipitation after a duration of at least one week.

Clause 48. The composition of clause 47, which exhibits no precipitation after a duration of at least eight weeks.

Clause 49. The composition of clause 48, which exhibits no precipitation after a duration of at least three months.

Clause 50. The composition of any one of clauses 2-49, wherein the composition is heat stable when subjected to a temperature of 135° C. for 45 minutes.

Clause 51. The composition of any one of clauses 1, 6-23, 39-40 and 43-44, wherein the composition is in a solid dry state.

Clause 52. A composition comprising caffeine, sodium benzoate, and lactic acid.

Clause 53. The composition of clause 52, further comprising water.

Clause 54. The composition of clause 53, wherein the composition is in the form of an aqueous solution.

Clause 55. The composition of any one of clauses 53-54, wherein the composition is stable against formation of precipitates, is heat stable when subjected to a temperature of 135° C. for 45 minutes, or both.

Clause 56. A pharmaceutical composition comprising a composition of any one of clauses 1-55 and optionally one or more pharmaceutically acceptable excipients.

Clause 57. The pharmaceutical composition of clause 56, wherein the amount of caffeine is from about 250 mg to about 1200 mg.

Clause 58. The pharmaceutical composition of clause 57, wherein the amount of caffeine is about 750 mg.

Clause 59. The pharmaceutical composition of any one of clauses 56-58, wherein the one or more pharmaceutically acceptable excipients comprise one or more preservatives, one or more antioxidants, one or more buffers, or any combination of two or more of these.

Clause 60. A kit comprising caffeine, sodium benzoate, and one or more of lactic acid, acetic acid, aspartic acid and glutamic acid.

Clause 61. The kit of clause 60, which comprises lactic acid.

Clause 62. The kit of any one of clauses 60-61, further comprising water.

Clause 63. A method of accelerating emergence from anesthesia or countering the effects of anesthesia comprising administering to a patient undergoing a medical procedure with an anesthesia agent a pharmaceutically effective amount of a composition of any one of clauses 1-59.

Clause 64. The method of clause 63, wherein the administration is parenteral.

Clause 65. The method of clause 63, wherein the administration is intravenous, intramuscular, subcutaneous, buccal, or oromucosal.

Clause 66. The method of any one of clauses 63-65, wherein the administration is through a bolus dose.

Clause 67. The method of any one of clauses 63-65, wherein the administration is through infusion or a drip line.

Clause 68. The method of any one of clauses 63-67, wherein the administration occurs concurrently with the administration of an anesthesia agent to the patient.

Clause 69. The method of any one of clauses 63-67, wherein the administration occurs after the delivery of an anesthesia agent to the patient.

Clause 70. The method of any one of clauses 63-67, wherein the administration occurs before the delivery of an anesthesia agent to the patient.

Clause 71. The method of any one of clauses 63-70, wherein the anesthesia agent is one or more of halothane, isoflurane, enflurane, desflurane, sevoflurane, etomidate, lidocaine, magnesium, forskolin, theophylline, methylphenidate, preladenant, fentanyl, remifentanil, morphine, hydromorphone, and propofol.

Clause 72. The method of any one of clauses 63-70, wherein the anesthesia agent is propofol.

Clause 73. The method of any one of clauses 63-72, which comprises accelerating emergence from anesthesia, wherein the emergence from anesthesia is at least 15 minutes faster than without administering a composition of any one of clauses 1-59.

Clause 74. The method of any one of clauses 63-72, which comprises accelerating emergence from anesthesia, wherein the emergence from anesthesia is at least 30 minutes faster than without administering a composition of any one of clauses 1-59.

Clause 75. The method of any one of clauses 63-74, which comprises countering the effects of anesthesia, wherein the method of countering the effects of anesthesia is reversing the effects of anesthesia.

Clause 76. A parenteral pharmaceutical composition comprising a composition of any one of clauses 1-59.

Clause 77. The parenteral pharmaceutical composition of clause 76, wherein the dose volume is from about 2 to about 8 mL.

Clause 78. The parenteral pharmaceutical composition of any one of clauses 76-77, which is configured for intravenous administration.

Clause 79. The parental pharmaceutical composition of any one of clauses 76-77, which is configured for intramuscular or subcutaneous routes.

Clause 80. Use of a pharmaceutical composition comprising an aqueous solution of caffeine, sodium benzoate, and one or more of lactic acid, acetic acid, aspartic acid and glutamic acid for treating a host mammal undergoing a medical procedure with an anesthesia agent.

Clause 81. The use of clause 80, wherein the pharmaceutical composition further comprises one or more pharmaceutically acceptable excipients.

Clause 82. The use of any one of clauses 80-81, wherein the pharmaceutical composition is not an oral dosage form.

Clause 83. The use of any one of clauses 80-82, wherein the pharmaceutical composition is an intravenous dosage form and the host mammal is human.

Clause 84. Use of a pharmaceutical composition comprising an aqueous solution of caffeine, sodium benzoate, and one or more of lactic acid, acetic acid, aspartic acid and glutamic acid for accelerating emergence from anesthesia or countering the effects of anesthesia in a host mammal.

Clause 85. The use of clause 84, wherein the mammal is human.

Clause 86. The use of any one of clauses 84-85, wherein the pharmaceutical composition is not an oral dosage form.

Clause 87. The use of any one of clauses 84-86, wherein the pharmaceutical composition is an intravenous dosage form and the host mammal is human.

Clause 88. The use of any one of clauses 84-87, wherein the method of countering the effects of anesthesia is reversing the effects of anesthesia.

Clause 89. The use of any one of clauses 80-82, 84, 86 and 88, wherein the host mammal is a non-human mammal.

Clause 90. A process for preparing a caffeine pharmaceutical composition comprising combining water with sodium benzoate to form a solution; treating the sodium benzoate solution with caffeine, and then adding one or more of lactic acid, acetic acid, aspartic acid and glutamic acid to form a caffeine pharmaceutical composition.

Clause 91. The process of clause 90, wherein the one or more of lactic acid, acetic acid, aspartic acid and glutamic acid is in an aqueous solution.

Clause 92. A caffeine pharmaceutical composition prepared by the process of any one of clauses 90-91.

Clause 93. The caffeine pharmaceutical composition of clause 92, further comprising one or more pharmaceutically acceptable excipients.

Clause 94. The caffeine pharmaceutical composition of any one of clauses 92-93, wherein the pharmaceutical composition is an intravenous dosage form.

Clause 95. The caffeine pharmaceutical composition of any one of clauses 92-93, which is configured for intravenous, intramuscular, subcutaneous, buccal, or oromucosal delivery.

Clause 96. A method of treating a patient suffering from opioid intoxication or overdose comprising administering to the patient a pharmaceutically effective amount of a composition of any one of clauses 1-59.

Clause 97. The method of clause 96, wherein the administration is parenteral.

Clause 98. The method of clause 96, wherein the administration is intravenous, intramuscular or subcutaneous.

Clause 99. The method of any one of clauses 96-98, wherein the administration is through a bolus dose.

Clause 100. The method of any one of clauses 96-98, wherein the administration is via infusion or a drip line.

Clause 101. The method of any one of clauses 96-100, wherein the patient is co-administered naloxone.

Clause 102. The method of clause 101, wherein naloxone is co-administered before administering the composition of any one of clauses 1-59.

Clause 103. The method of clause 101, wherein naloxone is co-administered during administering the composition of any one of clauses 1-59.

Clause 104. The method of clause 101, wherein naloxone is co-administered after administering the composition of any one of clauses 1-59.

Clause 105. The method of any one of clauses 96-104, wherein the opioid is oxycodone, hydrocodone, morphine, methadone, fentanyl or heroin.

Clause 106. Use of a pharmaceutical composition comprising caffeine, sodium benzoate, and one or more of lactic acid, acetic acid, aspartic acid and glutamic acid for treating a host mammal suffering from opioid intoxication or overdose.

Clause 107. The use of clause 106, wherein the pharmaceutical composition further comprises water.

Clause 108. The use of any one of clauses 106-107, wherein the pharmaceutical composition further comprises one or more pharmaceutically acceptable excipients.

Clause 109. The use of any one of clauses 106-108, wherein the pharmaceutical composition is not an oral dosage form.

Clause 110. The use of any one of clauses 106-109, wherein the pharmaceutical composition is an intravenous dosage form and the host mammal is human.

Clause 111. A beverage, food or candy, which comprises a composition of any one of clauses 1-55.

Clause 112. The beverage, food or candy of clause 111, which is a baked food made from flour.

Clause 113. The beverage, food or candy of clause 112, which is a cake, pastry, doughnut or cookie.

Clause 114. The beverage, food or candy clause 111, which is chocolate.

Clause 115. The beverage, food or candy of clause 111, which is gum or a breath freshener.

Clause 116. The beverage, food or candy of clause 111, which is a beverage, wherein the beverage is a carbonated beverage, tea, coffee or energy drink.

Clause 117. A composition comprising caffeine, sodium benzoate, and one or more C₂ to C₅ carboxylic acids.

Clause 118. A composition comprising caffeine, sodium benzoate, and one or more C₄ to C₅ amino acids.

Clause 119. A composition comprising caffeine, sodium benzoate, and one or more C₂ to C₅ carboxylic acid alcohols.

Clause 120. A composition comprising caffeine, sodium benzoate, and one or more C₂ to C₅ dicarboxylic acids.

Clause 121. The composition of any one of clauses 117-120, further comprising water.

Clause 122. The composition of clause 121, wherein the composition is in the form of a solution.

### EXAMPLES

### Example 1—Material, Equipment, and Software

The following material, equipment, and software were used in Examples 2-3 of the disclosure.

Materials:
Distilled water (Meijer, lot 39-222, exp Oct. 31, 2020), (also known as Water)
Lactic acid in aqueous solution ≥85% ACS Regent (EMD Millipore Corp, lot 56169630), (also known as Lactic acid)
Caffeine (Acros, lot A0405347), 98.5%
Sodium benzoate (TCI, lot FAPHB-JF), >98%

Equipment:
Denver Instrument Company A-250 balance
Eppendorf Research 1000 pipette, range 0.100-5.000 mL
Sonicator (Model O Conx. Jewelry Cleaner)
Escali model PRS00S scale
Mettler Toledo AG104 balance
VWR Scientific Symphony SB21 pH meter, equipped with Cole-Parmer glass electrode, BNC (05991-61)
VELP Scientifica stir plate

### Example 2—Preparation of Caffeine, Sodium Benzoate, and Lactic Acid Compositions

Several hundred experiments were run in a design of experiment model to identify solubility-enhanced caffeine in sodium benzoate. Most did not produce caffeine that remained soluble in solution for a desired period of time. Several positive results are reported herein. Compositions were made by the following process with amounts of materials used set forth in Tables 2 and 3. A magnetic stir bar was placed into a 20 mL scintillation vial and the vial was placed onto a stir plate; all additions were made while stirring. Approximately half of the water was added to the vial followed by sodium benzoate and stirred until dissolution. The appropriate weight of caffeine was added to the vial, and the remainder of the water was added to the vial. The lactic acid was then added to the vial, and the vial was capped.

TABLE 2

| | | | | | | Caffeine |
|---|---|---|---|---|---|---|
| | | Sodium | | Lactic | | dissolved |
| | Water | benzoate | Caffeine | acid | Total | in solution |
| Composition | (mL) | (mg) | (mg) | (mL) | (mL) | (mg/mL) |
| RP1-49-2 | 9.768 | 2411.8 | 2500.1 | 0.232 | 10.000 | 250 |
| RP1-49-3 | 9.760 | 2504.8 | 2500.4 | 0.240 | 10.000 | 250 |
| RP1-49-4 | 9.751 | 2597.4 | 2500.1 | 0.249 | 10.000 | 250 |
| RP1-49-5 | 9.742 | 2690.3 | 2500.1 | 0.258 | 10.000 | 250 |

TABLE 3

| | Equivalents | | | |
|---|---|---|---|---|
| | Sodium | Equivalents | Equivalents | |
| Composition | benzoate | Caffeine | Lactic acid | pH |
| RP1-49-2 | 1.300 | 1.000 | 0.195 | 5.86 |
| RP1-49-3 | 1.350 | 1.000 | 0.202 | 5.87 |
| RP1-49-4 | 1.400 | 1.000 | 0.209 | 5.94 |
| RP1-49-5 | 1.450 | 1.000 | 0.217 | 5.92 |

Samples of each composition were refrigerated at −2° C. and have remained clear for more than 8 weeks.

### Example 3—Additional Preparations of Caffeine, Sodium Benzoate, and Lactic Acid Compositions

Sample: RP1-57-5 (235 mg:235 mg, Caffeine: Sodium Benzoate)

2,350.2 mg of sodium benzoate was accurately weighed and added to a 20 mL scintillation vial containing a stir bar. 2350.1 mg caffeine was accurately weighed and added to the same vial. 10 mL distilled water was subsequently pipetted into the vial and the vial was capped. The vial was placed onto a magnetic stir plate and allowed to stir at 300 rpm until all solids were completely dissolved, resulting in a solution with 235 mg/caffeine per mL of solution. The pH was measured to be 8.01.

Sample: RP1-60-1

A magnetic stir bar was placed into a 20 mL scintillation vial and the vial was placed onto a stir plate. While stirring at 300 rpm, 5 mL distilled water was pipetted into the vial. 2,597.5 mg of sodium benzoate was accurately weighed and added to the vial; the vial was allowed to stir until the sodium benzoate dissolved completely. 2,500.3 mg of caffeine was accurately weighed and added to the same vial. 1.25 mL distilled water was added to the vial followed immediately by 249 μL of an 88.47% by weight lactic acid solution in water and the vial was capped. An additional 1.0 mL distilled water was added the next day and 0.5 mL on the subsequent day. The vial was allowed to stir at 300 rpm until all solids were completely dissolved, resulting in a solution with 313 mg caffeine per mL of solution. The pH was measured to be 5.87.

Sample: RP1-60-2

A magnetic stir bar was placed into a 20 mL scintillation vial and the vial was placed onto a stir plate. While stirring at 300 rpm, 5 mL distilled water was pipetted into the vial. 2,690.2 mg of sodium benzoate was accurately weighed and added to the vial; the vial was allowed to stir until the sodium benzoate dissolved completely. 2,500.1 mg of caffeine was accurately weighed and added to the same vial. 1.25 mL distilled water was added to the vial followed immediately by 258 μL of an 88.47% by weight lactic acid solution in water and the vial was capped. An additional 1.0 mL distilled water was added the next day and 0.25 mL on the subsequent day. The vial was allowed to stir at 300 rpm until all solids were completely dissolved, resulting in a solution with 322 mg caffeine per mL of solution. The pH was measured to be 5.94.

Sample: RP1-62-4 (396 mg/mL Caffeine, 18% Lactic Acid to Sodium Benzoate)

A magnetic stir bar was placed into a 20 mL scintillation vial and the vial was placed onto a stir plate. While stirring at 300 rpm, 5 mL distilled water was pipetted into the vial. 2,690.5 mg of sodium benzoate was accurately weighed and added to the vial; the vial was allowed to stir until the sodium benzoate dissolved completely. 2,500.2 mg of caffeine was accurately weighed and added to the same vial. 1 mL distilled water was added to the vial followed immediately by 310 μL of an 88.47% by weight lactic acid solution in water and the vial was capped. The vial was allowed to stir at 300 rpm until all solids were completely dissolved, resulting in a solution with 396 mg of caffeine per mL of solution. The pH was measured to be 5.91.

The solids in all samples remained dissolved for at least three weeks.

### Example 4—Samples RP1-65-1, RP1-65-2 and RP1-68-1

Sample RP1-62-4 was recreated at both the 10 ml scale and a 100 ml scale (two samples). The order of addition was half the water, sodium benzoate, stir to dissolve, caffeine, remaining water, lactic acid. The second addition of water was used to rinse any remaining solids from the glassware. The larger scale samples were created in 250 ml Pyrex glass media bottles containing large stir bars. The samples were removed from the stir plates and stored on the lab bench under ambient conditions.

Table 4 provides the formulation details for the compositions prepared.

TABLE 4

| | | Sodium | Lactic | |
|---|---|---|---|---|
| | Caffeine | benzoate | acid | Water |
| Composition | (mg) | (mg) | (ml) | (ml) |
| RP1-65-1 | 2500.0 | 2690.4 | 0.310 | 9.690 |
| RP1-65-2 | 25003.0 | 269050 | 3.100 | 96.900 |
| RP1-68-1 | 25001.4 | 269044 | 3.100 | 96.900 |

The pH values were measured after approximately two weeks as 5.78, 5.77 and 5.77 for compositions RP1-65-1, RP1-65-2 and RP1-68-1, respectively. Caffeine solubility was 250 mg/ml for the compositions. All compositions remained clear for over three months.

### Example 5—Compositions Comprising Acetic Acid, Aspartic Acid or Glutamic Acid

Several compositions that achieved 250 mg/ml caffeine with long term stability are summarized below in Table 5.

TABLE 5

| | | | | | | Lactic | |
|---|---|---|---|---|---|---|---|
| | | Sodium | Lactic | | Molar equivalents | acid to | |
| | Caffeine | benzoate | acid | Water | caffeine:sodium | sodium | |
| Composition | (mg) | (mg) | (ml) | (ml) | benzoate:lactic acid | benzoate | pH |
| RP1-49-2 | 2500.1 | 2411.8 | 0.232 | 9.768 | 1:000 to 1.300 to 0.195 | 15% | 5.86 |
| RP1-49-3 | 2500.4 | 2504.8 | 0.240 | 9.760 | 1:000 to 1.350 to 0.202 | 15% | 5.87 |
| RP1-49-4 | 2500.1 | 2597.4 | 0.249 | 9.751 | 1:000 to 1.400 to 0.209 | 15% | 5.94 |
| RP1-49-5 | 2500.1 | 2690.3 | 0.258 | 9.742 | 1:000 to 1.450 to 0.217 | 15% | 5.92 |
| RP1-65-1 | 2500.0 | 2690.4 | 0.310 | 9.690 | 1:000 to 1.450 to 0.261 | 18% | 5.78 |

These formulations were recreated, replacing lactic acid with acetic acid (glacial acetic acid, VWR Chemicals), aspartic acid (D-Aspartic acid (99+%)) or glutamic acid (L-glutamic acid 99%, Aldrich Chemical Co. Inc.). All samples were created in 20 ml scintillation vials, adding approximately half the water first, followed by sodium benzoate (stir until dissolved), then caffeine, the remainder of the water, and the acid. If the acid to be added was solid, the second addition of water was added at the end instead of after the caffeine.

The acetic acid samples (Table 6) quickly dissolved within 15 minutes, had essentially the same pH as the lactic acid samples, and remained dissolved for more than three months.

TABLE 6

| | | Sodium | Acetic | | |
|---|---|---|---|---|---|
| | Caffeine | benzoate | acid | Water | |
| Composition | (mg) | (mg) | (ml) | (ml) | pH |
| RP1-66-1 | 2500.1 | 2411.7 | 0.144 | 9.856 | 5.87 |
| RP1-66-2 | 2500.5 | 2504.9 | 0.149 | 9.851 | 5.87 |
| RP1-66-3 | 2500.4 | 2597.6 | 0.154 | 9.846 | 5.84 |
| RP1-66-4 | 2500.3 | 2690.4 | 0.160 | 9.840 | 5.85 |
| RP1-66-5 | 2500.1 | 2690.2 | 0.192 | 9.808 | 5.78 |

The aspartic acid samples (Table 7) dissolved relatively quickly. After about a month, all solutions were slightly yellow. After three months, the first two samples had formed precipitates and the last three samples were still clear.

TABLE 7

| | | Sodium | Aspartic | | |
|---|---|---|---|---|---|
| | Caffeine | benzoate | acid | Water | |
| Composition | (mg) | (mg) | (mg) | (ml) | pH |
| RP1-67-1 | 2500.4 | 2411.9 | 334.4 | 10.000 | 5.77 |
| RP1-67-2 | 2500.4 | 2504.7 | 346.7 | 10.000 | 5.77 |
| RP1-67-3 | 2500.1 | 2597.7 | 358.3 | 10.000 | 5.78 |
| RP1-67-4 | 2500.2 | 2690.3 | 372.2 | 10.000 | 5.79 |
| RP1-67-5 | 2500.3 | 2690.3 | 447.2 | 10.000 | 5.68 |

The glutamic acid samples (Table 8) dissolved relatively quickly, within the same day or the next day. After three months, the first sample had formed precipitates and all other samples were still clear.

TABLE 8

| | | Sodium | Glutamic | | |
|---|---|---|---|---|---|
| | Caffeine | benzoate | acid | Water | |
| Composition | (mg) | (mg) | (mg) | (ml) | pH |
| RP1-67-6 | 2500.3 | 2411.9 | 369.7 | 10.000 | 5.77 |
| RP1-67-7 | 2500.5 | 2504.9 | 382.9 | 10.000 | 5.78 |
| RP1-67-8 | 2500.5 | 2597.7 | 395.9 | 10.000 | 5.80 |
| RP1-67-9 | 2500.5 | 2690.1 | 411.5 | 10.000 | 5.80 |
| RP1-67-10 | 2500.5 | 2690.6 | 494.8 | 10.000 | 5.71 |

### Example 6—Batch Production Method

The following materials and equipment were used in a batch production method for making exemplary compositions comprising caffeine, sodium benzoate and water.

### Materials:

Caffeine
Sodium benzoate
Water
Lactic acid in aqueous solution (˜88.5%)

### Equipment:

Glass media bottle
Spatulas
Weighing containers
Magnetic stir bar
Glass funnels
Balance
Pipette, 5000 μL
Stir plate

The batch production method includes procedure described in the following steps.

A clean magnetic stir bar was placed into a clean 250 mL glass media bottle, the bottle placed onto a stir plate (300 rpm) and 30 mL of water (one third the total water volume) was accurately measured, volume recorded and added to the bottle.

A clean glass funnel was placed into the top of the bottle opening.

Sodium benzoate (26.9044 g) was accurately weighed into an appropriate container, weight recorded and slowly added to the funnel using a clean spatula, making certain not to spill any of the powdered solids.

The funnel, spatula and weighing container were rinsed into the bottle with accurately measured 5 mL aliquots of water. A total of 30 mL of water was required for thorough rinsing.

Once the funnel was removed, the bottle was capped and allowed to stir until the sodium benzoate was completely dissolved. Once a clear solution resulted in the bottle, the cap was removed and a clean glass funnel was placed into the top of the bottle opening.

Caffeine was accurately weighed (25.0014 g) into an appropriate container, weight recorded and slowly added to the funnel using a clean spatula, making certain not to spill any of the powdered solids. The funnel, spatula and weighing container were rinsed into the bottle with accurately measured 5 mL aliquots of water. A total of 30 mL of water was required for thorough rinsing. Once the funnel was removed, the remainder of the water (6.9 mL) was accurately measured, volume recorded and added to the bottle.

Lactic acid (3.1 mL) was accurately measured, added to the bottle immediately, and the bottle was again capped and allowed to stir (300 rpm) until all solids completely dissolved.

### Example 7—Composition Stability after Processing at 135° C. for 45 Minutes

A 250 mg/mL caffeine, 18% lactic acid:sodium benzoate composition was diluted 10,000-fold, added to an amber HPLC vial, tightly capped and placed into an oven at 135° C. After 45 minutes, the vial was removed and analyzed by HPLC.

The HPLC chromatogram for the unheated sample displayed appropriate peak shape with no overlapping peaks. A peak with retention time of 4.209 minutes corresponded to caffeine while a peak with retention time of 7.545 minutes corresponded to sodium benzoate. The HPLC results for the chemical stability evaluation of the composition revealed no additional extraneous peaks following exposure to 135° C. for 45 minutes.

### Example 8—Prophetic

A 200-pound male who has overdosed on an opioid is treated with a 2.5 mg caffeine/kg of body weight to 15 mg caffeine/per kg of body weight a caffeine composition of the disclosure which is administered by IV and co-administered with naloxone. The combination reverses general acute respiratory depression and or recurrent respiratory depression associated with opioid overdose and reduces the dose and frequency that would otherwise be given of naloxone.

1. A composition comprising caffeine, sodium benzoate, and one or more of lactic acid, acetic acid, aspartic acid and glutamic acid.

2. The composition of claim 1, further comprising water.

3. The composition of claim 2, wherein the composition is in the form of a solution.

4. A composition comprising:
an aqueous solution of caffeine and sodium benzoate, and
means for increasing the solubility of caffeine in the aqueous solution.

5-6. (canceled)

7. The composition of claim 1, which comprises lactic acid.

8-11. (canceled)

12. The composition of claim 1, wherein the molar ratio of caffeine to sodium benzoate is from about 1:1.0 to about 1:1.5.

13-17. (canceled)

18. The composition of claim 1, wherein the molar ratio of caffeine to the total moles of lactic acid, acetic acid, aspartic acid and glutamic acid is from about 1:0.1 to about 1:0.3.

19-22. (canceled)

23. The composition of claim 1, wherein the molar ratio of sodium benzoate to the total moles of lactic acid, acetic acid, aspartic acid and glutamic acid is from about 1:0.15 to about 1:0.18.

24. The composition of claim 2, wherein the pH of the composition is from about 5 to about 6.

25-29. (canceled)

30. The composition of claim 2, wherein the solubility of the caffeine is 240 mg/ml or greater.

31-36. (canceled)

37. The composition of claim 2, wherein the weight percent of caffeine is from about 15% to about 25%, the weight percent of sodium benzoate is from about 15% to about 25%, the weight percent of lactic acid is from about 1% to about 3%, and the weight percent of water is from about 50% to about 70%.

38-44. (canceled)

45. A composition of claim 1 comprising caffeine, sodium benzoate, lactic acid and water;
wherein the composition is in the form of an aqueous solution;
wherein the molar ratio of caffeine to sodium benzoate is from about 1:1.3 to about 1:1.45;
wherein the molar ratio of sodium benzoate to lactic acid is from about 1:0.15 to about 1:0.18; and
wherein the solubility of the caffeine in the aqueous solution is 250 mg/mL or greater.

46-49. (canceled)

50. The composition of claim 3, wherein the composition is heat stable when subjected to a temperature of 135° C. for 45 minutes.

51-55. (canceled)

56. A pharmaceutical composition comprising a composition of claim 1 and one or more pharmaceutically acceptable excipients.

57-62. (canceled)

63. A method of accelerating emergence from anesthesia or countering the effects of anesthesia comprising administering to a patient undergoing a medical procedure with an anesthesia agent a pharmaceutically effective amount of a composition of claim 1.

64. (canceled)

65. The method of claim 63, wherein the administration is intravenous, intramuscular, subcutaneous, buccal, or oromucosal.

66-75. (canceled)

76. A parenteral pharmaceutical composition comprising a composition of claim 1.

77-78. (canceled)

79. The parental pharmaceutical composition of claim 76, which is configured for intramuscular or subcutaneous routes.

80-95. (canceled)

96. A method of treating a patient suffering from opioid intoxication or overdose comprising administering to the patient a pharmaceutically effective amount of a composition of claim 1.

97-122.

123. A composition in the form of an aqueous solution and comprising caffeine at a solubility of greater than about 235 mg/mL in the solution, or
a composition comprising caffeine, sodium benzoate, and lactic acid, or
a kit comprising caffeine, sodium benzoate, and one or more of lactic acid, acetic acid, aspartic acid and glutamic acid, or
a process for preparing a caffeine pharmaceutical composition comprising combining water with sodium benzoate to form a solution; treating the sodium benzoate solution with caffeine, and then adding one or more of lactic acid, acetic acid, aspartic acid and glutamic acid to form a caffeine pharmaceutical composition, or
a composition comprising caffeine, sodium benzoate, and one or more C₂ to C₅ carboxylic acids, or
a composition comprising caffeine, sodium benzoate, and one or more C₄ to C₅ amino acids, or
a composition comprising caffeine, sodium benzoate, and one or more C₂ to C₅ carboxylic acid alcohols, or
a composition comprising caffeine, sodium benzoate, and one or more C₂ to C₅ dicarboxylic acids.
